(19) United States
(12) Patent Application Publication
(10) Pub. No.: US 2013/0213815 A1
(43) Pub. Date: Aug. 22, 2013

(54) Method of Fabricating a Nanochannel System for DNA Sequencing and Nanoparticle Characterization

(71) Applicants: Chao-Hung Steve Tung, Fayetteville, AR (US); Jin-Woo Kim, Fayetteville, AR (US)

(72) Inventors: Chao-Hung Steve Tung, Fayetteville, AR (US); Jin-Woo Kim, Fayetteville, AR (US)

(21) Appl. No.: 13/768,960

(22) Filed: Feb. 15, 2013

Related U.S. Application Data
(60) Provisional application No. 61/633,712, filed on Feb. 16, 2012.

(51) Int. Cl. C12Q 1/68

(52) U.S. Cl. CPC C12Q 1/6869; B82Y 40/00
USPC 205/114; 204/601; 204/451; 977/890; 977/924; 977/700

(57) ABSTRACT

A process for fabricating a nanochannel system using a combination of microelectromechanical system (MEMS) microfabrication techniques and atomic force microscopy (AFM) nanolithography. The nanochannel system, fabricated on either a glass or silicon substrate, has channel heights and widths on the order of single to tens of nanometers. The channel length is in the micrometer range. The nanochannel system is equipped with embedded micro or nanoscale electrodes, positioned along the length of the nanochannel for electron tunneling based characterization of nanoscale particles in the channel. Anodic bonding is used to cap off the nanochannel with a cover chip.

### CROSS-REFERENCE TO RELATED APPLICATIONS

This application claims the benefit of U.S. Provisional Application No. 61/633,712, entitled “Method of Fabricating a Nanochannel System for DNA Sequencing and Nanoparticle Characterization” and filed on Feb. 16, 2012. The complete disclosure of said provisional patent application is hereby incorporated by reference.

### STATEMENT REGARDING FEDERALLY SPONSORED RESEARCH OR DEVELOPMENT

Not applicable.

### BACKGROUND OF THE INVENTION

The present invention relates to a nanochannel system including a nanofluidic device for rapid DNA sequencing with single-base resolution and single nanoparticle characterization based on electron tunneling, and in particular, to a method of fabrication of such a nanochannel by means of microelectromechanical system (MEMS) microfabrication techniques and atomic force microscopy (AFM) nanolithography.

### BRIEF SUMMARY OF THE INVENTION

The present invention is directed to a process for fabricating a nanochannel system using a combination of microelectromechanical system (MEMS) microfabrication techniques and atomic force microscopy (AFM) nanolithography. The process includes (1) a micropatterning step to form at least one electrode on a substrate, (2) a micropatterning step to form a microchannel having inlet and outlet portions on the substrate, (3) an AFM nanolithography step to form a nanochannel connecting the inlet and outlet portions of the microchannel and to dissect the electrode, and (4) an anodic bonding step to bond a cover chip onto the substrate so that the nanochannel is closed.

The nanochannel system is a nanotechnology based rapid DNA sequencing technique that achieves sequencing without the use of lengthy sample pre-treatment and DNA replication currently used by other DNA sequencing techniques. The result is a much faster and cost-effective chip-based sequencing method that can benefit both the biomedical and DNA research communities. The device can sequence a single stand of DNA.

The nanochannel system is embedded with sensing electrodes to detect electrical signals of DNA bases. The electrodes are positioned to produce an electron tunneling system and to guide the DNA as a single strand without folds or loops through the sequencing process.

The nanochannel is fabricated by a nanomachining method that is both precise and easy to operate. The fabrication method does not require cleanroom processing and is therefore cheaper to operate than other semiconductor based techniques.

The nanochannel system, fabricated on either a glass or silicon substrate, has channel heights and widths on the order of single to tens of nanometers. The nanochannel length is in the micrometer range. The nanochannel system is equipped with embedded micro or nanoscale electrodes, positioned along the length of the channel for electron tunneling based characterization of nanoscale particles in the channel. Electron tunneling is quantum phenomenon where an electron ‘tunnels’ through a potential barrier that repels a classical particle with the same energy. In the nanochannel system, the embedded electrodes measure the tunneling current of the nanoparticles as they translocate through the nanochannel. The nanochannel system is particularly suited for DNA sequencing. To accomplish this, individual DNA strands are electrically pulled through the nanochannel, where the DNAs translocate at a lower speed than in a nanopore due to high viscous drag, and the bases in the DNA strand are characterized by their corresponding electron tunneling current in the transverse direction. This method of DNA characterization is expected to yield a much higher temporal and spatial resolution than the nanopore approach.

Particularly important features associated with the invention are:

AFM based nanolithography together with anodic bonding can be used to fabricate nanochannel systems.

Micro to nanoscale electrodes can be fabricated along the AFM nanochannel for electrical characterization of nanoscale particles in the channel.

Fabrication of the nanochannel system is relatively fast and easy by combining MEMS microfabrication with AFM nanolithography.

Continuous nanoscale liquid flow can be maintained in the nanochannel.

The nanochannel system with embedded electrodes can be used to characterize the electron tunneling current of translocating nanoparticles.

The nanochannel system with embedded electrodes can be used to sequence single-stranded DNA with single-base resolution.

These and other features, objects and advantages of the present invention will become better understood from a consideration of the following detailed description of the preferred embodiments and appended claim in conjunction with the drawings as described following.

### BRIEF DESCRIPTION OF THE SEVERAL VIEWS OF THE DRAWINGS

FIG. 1 is a plan view of a microfabricated silicon chip with inlet and outlet reservoirs, microchannel, and a plurality of microelectrodes.

FIG. 2 is a close-up view of the section of FIG. 1 where a nanochannel is to be machined.

FIG. 3 is a close-up view of FIG. 2 showing a nanochannel machined by AFM nanolithography (30 μm long, 20 nm deep, and 100 nm wide).

FIG. 4 is a schematic illustration of a setup for AFM machining of a nanochannel.

FIG. 5 is a schematic illustration of a setup for anodic bonding of a cover chip to a silicon substrate.

FIG. 6 illustrates an experimental setup for driving negatively-charged FluoSpheres® through a nanochannel system by positive electric field while measuring the electrical current of the transverse electrodes.

FIGS. 7(a) and 7(b) are fluorescent images (10×) of the silicon nanochannel system before (FIG. 7(a)) and after (FIG. 7(b)) negatively-charged FluoSpheres® are driven through the nanochannel by a positive electric field.

FIG. 8 is a graph of the instantaneous tunneling current measurement of translocating nanoparticles.

### DETAILED DESCRIPTION OF THE INVENTION

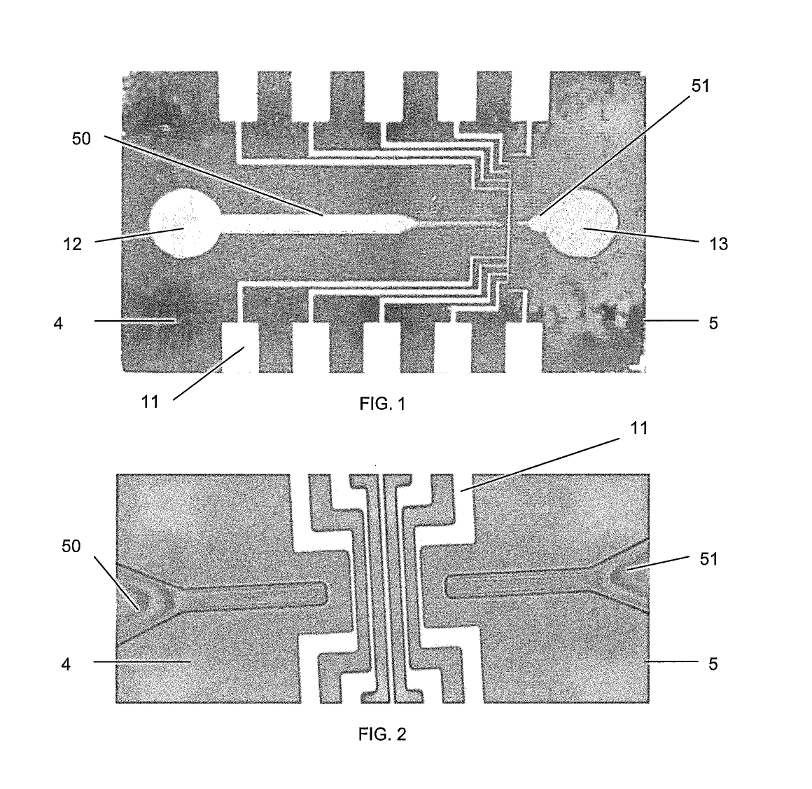

The present invention uses AFM nanolithography in conjunction with MEMS microfabrication techniques to create a nanochannel system with integrated microelectrodes 11. The fabrication process involves two micropatterning steps (one to form at least one electrode 11 and another to form a microchannel in two portions—an inlet portion 50 and an outlet portion 51), one AFM nanolithography step, and one chip bonding step. The fabrication process for a silicon nanochannel system begins with the patterning of the microchannel inlet portion 50 and outlet portion 51 and at least one electrode on a substrate, such as a silicon chip 5, as shown in FIGS. 1 and 2. The electrodes 11 may be microelectrodes as shown in FIGS. 1-3 or nanoelectrodes. The electrodes may be formed of various materials known to those skilled in the art, including Cr/Au or Pt/Ti. A plurality of electrodes 11 are desirable.

FIG. 1 is an enlarged picture of a silicon chip 5 fabricated by a MEMS process. The microchannel inlet portion 50 may include an inlet microreservoir 12 and the microchannel 51 may include an outlet microreservoir 13. The microchannel inlet portion 50, inlet microreservoir 12, microchannel 51 and outlet microreservoir 13 are all desirably about 20-pm deep. The microchannel portions 50, 51 along with the inlet microreservoir 12 and the outlet microreservoir 13 serve as the inlet and outlet for the nanochannel 30. The electrodes 11 are desirably about 40-nm thick and reside on top of a 500-nm thick silicon oxide layer 4. High-temperature Pt as the electrode material allows thinner electrodes compatible with the high temperatures and voltage of the anodic bonding step; however, Au electrodes may also be compatible with the anodic bonding step where the electrodes are thicker in the range of about 40 nm to about 100 nm.

FIG. 2 is a close-up picture of the location on the silicon chip 5 where the nanochannel 30 is machined. In this embodiment, five 1-μm wide parallel microelectrodes 11 cross the path of the nanochannel 30. As the nanochannel 30 is machined, each microelectrode 11 is dissected into two matching microelectrodes 11 bordering the outline of the nanochannel 30. With these dissected microelectrodes 11, it becomes possible to measure the transverse electrical impedance of the nanochannel 30 at five distinct locations in the longitudinal direction. When needed, external bridge-type circuits (not shown) can be added to the system to monitor the instantaneous conductivity of the nanochannel 30 as a way to track the movement of a nanoscale object inside the nanochannel 30.

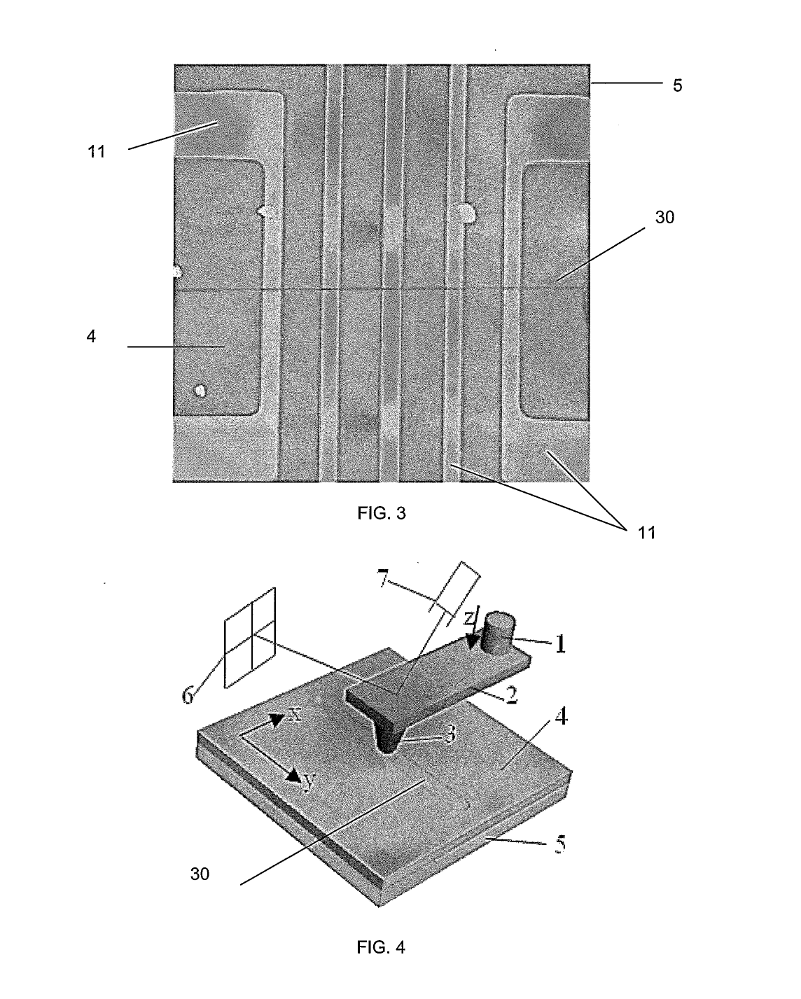

The nanochannel 30 is machined mechanically between the inlet 50 and outlet portions 51 of the microchannel using AFM nanolithography by means of a setup such as that shown in FIG. 4. A diamond probe tip 3 with a large spring constant and a nanoscale tip radius serves as the cutting tool. A calibration process is carried out in advance to establish the relationship between the tip control parameters such as force and speed, and the resultant dimensions of the nanochannel 30. As the nanochannel 30 is machined, each of the parallel microelectrodes 11 between the microchannels 10 is dissected into two matching tunneling microelectrodes 11 separated by the width of the nanochannel 30 as shown in FIG. 3.

In the nanochannel system shown in FIG. 3, the nanochannel 30 on the silicon chip 5 was mechanically machined in a Dimension 3100 AFM (Veeco Inc., CA) controlled by a Nanoscope IIla controller. The AFM probe used was an all-diamond nanoindenting tip 3 (PDNISP from Veeco) with a calibrated spring constant of 215 N/m and a nominal tip radius of 40 nm. The tip 3 is mounted on a cantilever 1 which is actuated by piezoelectric tubes (PZT) 1. FIG. 4 demonstrates the basic layout of the AFM machining method. In this method, the AFM tip 3 is pressed against the silicon oxide surface layer 4 of the silicon chip 5 with a constant force (by automatically adjusting the PZT 1 to keep the vertical deflection as sensed by the position sensing device (PSD) 6 constant and then translated along a preplanned path on the surface. Prior research to determine the relationship between the AFM control parameters and the resultant nanochannel dimensions is described in Z. Q. Wang, S. Tung, N. D. Jiao, et al., “Nanochannels on silicon oxide surface fabricated by atomic force microscopy,” Proceedings of the 2010 5th IEEE international conference on Nano/Micro Engineered and Molecular Systems, Jan. 20-23, 2010, Xiamen, China, pp. 630-633, 2010. A vertical deflection signal of 4.0V and a translation speed of 1 μm/s have been found to be acceptable in the practice of the present invention.

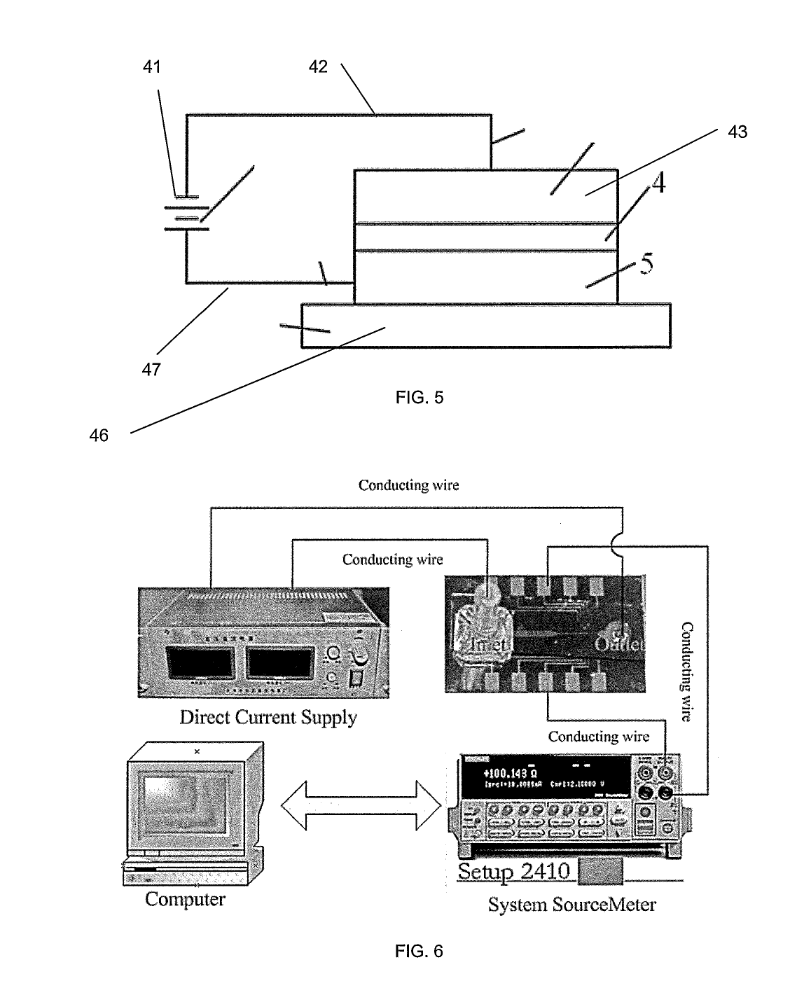

Once the nanochannel 30 is formed, the substrate chip 5 is capped off by a matching Pyrex cover chip to form a closed nanochannel 30 through anodic bonding. While Pyrex is the preferred material for use in the anodic bonding step, other anodic bonding materials and techniques as known to those skilled in the art may be used on the practice of the present invention. Anodic bonding is a technique to hermetically seal a substrate by bonding a cover chip to the substrate using a combination of heat and a strong electrostatic field. FIG. 5 is a schematic illustration of a setup for anodic bonding of the cover chip 43 to the silicon substrate 5.

The MEMS silicon substrate 5 with the AFM-machined nanochannel 30 was sealed off by a matching Pyrex cover chip 43 through anodic bonding. The 500-μm thick silicon substrate 5 was placed on a hot plate 6 and linked to the anode of a voltage-adjustable direct current supply 41. The Pyrex cover chip 43 (0.5 mm thick) with pre-drilled through holes over the inlet 12 and outlet microreservoirs 13 was placed on top of the silicon substrate 5 and linked to the cathode of the current supply 41. The hot plate 6 was maintained at a temperature to 550° C. At this plate temperature, the surface temperature of the silicon substrate 5 was measured as 420° C. by an infrared radiation thermometer. The anodic bonding process was performed at a voltage of 600V. The current supply showed the current to be between 0.2 and 0.4 mA at the beginning of the process. After about 20 minutes, the current dropped to about 0.01 mA at which point the bonding process was terminated.

A custom-built anodic bonding platform for performing the anodic bonding step included a 0.3-mm thick graphite disk (not shown) between the hotplate 6 and the silicon chip 5 to provide a uniform temperature distribution in the silicon chip. A 1 mm thick aluminum pressing block (not shown) on top of the Pyrex cover chip 43 ensured a good physical contact between the Pyrex cover chip 43 and the silicon substrate 5. The bonded chip was provided with a microfluidic connector (not shown) to the inlet reservoir 12 through the pre-drilled hole in the cover chip 43. Another pre-drilled hole over the outlet reservoir 13 provided an outlet to the nanochannel 30.

FIG. 6 shows an experimental setup for driving negatively-charged FluoSpheres® through the nanochannel 30 by a positive electric field while measuring the electrical current of the transverse electrodes 11. 20-nm carboxyl-modified FluoSpheres® (F-8787 from Invitrogen) were translocated through the nanochannel system through the use of an externally applied electric field. Since the FluoSpheres® are negatively charged, a positive voltage at the outlet reservoir 13, if high enough, tends to pull the nanobeads from the negatively-biased inlet reservoir 12 to the outlet reservoir 13 through the nanochannel 30. Once initiated, the nanobead flow is monitored by the transverse electrical current across the pairs of electrodes 11 positioned along the nanochannel 30.

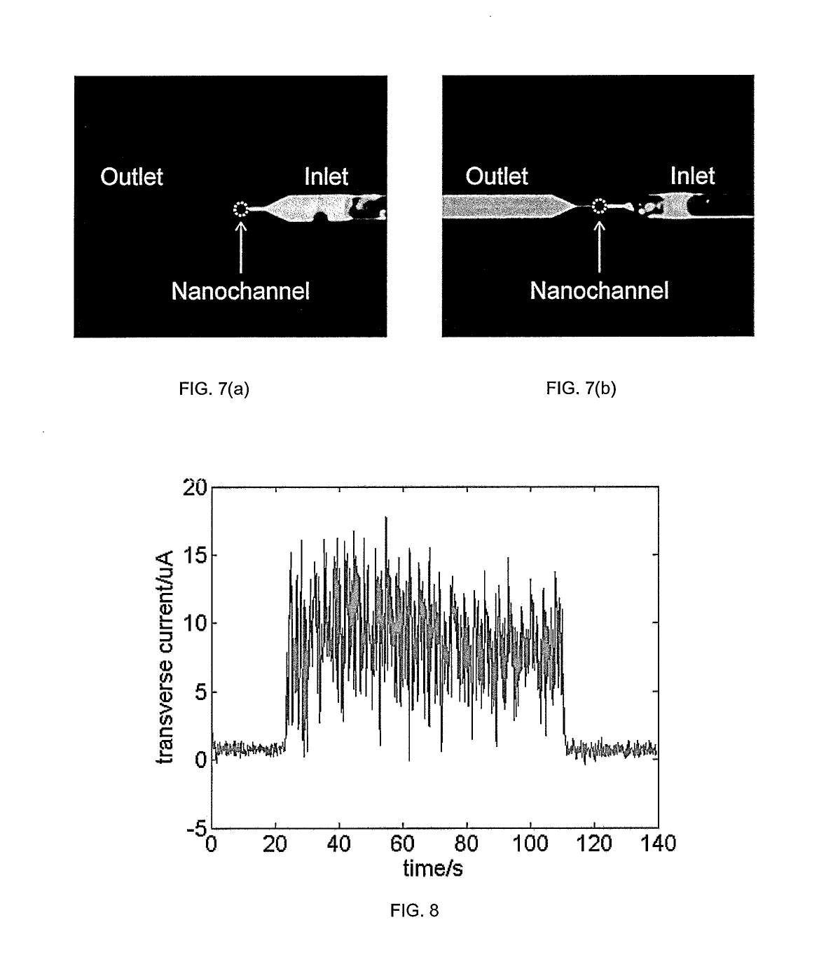

FIGS. 7(a) and 7(b) show fluorescent images (10×) of the silicon nanochannel system before (FIG. 7(a)) and after (FIG. 7(b)) negatively-charged FluoSpheres® are driven through the nanochannel 30 by a positive electric field. FIG. 7(a) demonstrates the filling of the inlet portion 50 of the microchannel by the FluoSpheres® suspension. Following this step, the outlet reservoir 13 is filled with 0.01 M phosphate-buffered saline (PBS) and a 10VDC bias is applied between the inlet and outlet reservoirs 12, 13. FIG. 7(b) demonstrates the result after a 3 min delay. The fluorescent pictures indicate that the upstream FluoSpheres® have been successfully translocated through the nanochannel 30 to the outlet reservoir 13 by the voltage bias.

FIG. 8 is a graph showing the instantaneous tunneling current measurement of translocating nanoparticles. FIG. 8 demonstrates the transverse electrical current measured by one pair of electrodes 11 in the nanochannel 30. When a voltage of 5VDC is applied, a large transverse current is obtained, indicating the flow of the conductive nanobeads significantly enhances the electrical conductivity of the nanochannel 30. Preliminary calculations based on quantum theories indicate the level of the current measured is consistent with the expected tunneling current of the nanobeads.

The nanochannel system fabricated by the method of the present invention has applications in DNA sequencing, protein analysis, virus detection, nanofluidic accelerometers, nanofluidic gyroscopes, nanoscale heat and mass transfer studies, and nano-filtration.

The AFM method for nanochannel formation does not require the expensive and time-consuming cleanroom techniques used by other nanochannel fabrication methods. In addition, the process is repeatable due to the precision control mechanism already in place in the AFM. Finally, the AFM method is scalable; multiple nanochannels can be machined simultaneously through the use of a multiple AFM tip setup currently being developed by AFM manufacturers. The AFM method is more cost-effective that other nanolithographic methods such as e-beam and focused ion beam techniques, which can only machine one channel at a time.

Insofar as the description above and the accompanying drawings disclose any additional subject matter that is not within the scope of the single claim below, the inventions are not dedicated to the public and the right to file one or more applications to claim such additional inventions is reserved.

Although a very narrow claim is presented herein, it should be recognized that the scope of this invention is much broader than presented by the claim. It is intended that broader claims will be submitted in one or more applications that claim the benefit of priority from this application.

The present invention has been described with reference to certain preferred and alternative embodiments that are intended to be exemplary only and not limiting to the full scope of the present invention.

What is claimed is:

1. A method of fabricating a nanochannel system comprising the steps of:
(a) micropatterning a substrate to form at least one electrode;
(b) micropatterning said substrate to form a first microchannel portion and a second microchannel portion;
(c) machining a nanochannel between said first microchannel portion and said second microchannel portion; and
(d) bonding a cover chip to said substrate.

2. The method of claim 1, wherein said substrate is a silicon chip.

3. The method of claim 1, wherein said at least one electrode is a microelectrode.

4. The method of claim 1, wherein said at least one electrode is a nanoelectrode.

5. The method of claim 1, wherein said substrate comprises a silicon oxide layer.

6. The method of claim 1, wherein said step of machining causes said at least one electrode to be dissected into at least two microelectrodes.

7. The method of claim 1, wherein said step of machining comprises the step of using atomic force microscopy nanolithography.

8. The method of claim 1, wherein said step of machining is performed by a cutting tool, wherein said cutting tool comprises a diamond probe tip with a large spring constant and a nanoscale tip radius, wherein said diamond probe tip is mounted on a cantilever.

9. The method of claim 1, wherein said cover chip is a Pyrex cover chip.

10. The method of claim 1, wherein said bonding is anodic bonding.

11. The method of claim 1, wherein said at least one electrode comprises five electrodes.

12. The method of claim 1, wherein said first microchannel portion is an inlet to said nanochannel and said second microchannel portion is an outlet from said nanochannel.

13. The method of claim 12, wherein said inlet comprises an inlet reservoir and said outlet comprises an outlet reservoir.

14. The method of claim 1, wherein said step of bonding a cover chip to said substrate comprises the steps of:
(a) placing said substrate on a hot plate;
(b) linking said substrate to an anode of a current supply;
(c) placing said cover chip on top of said substrate;
(d) linking said cover chip to a cathode of said current supply; and
(e) providing a temperature of said hot plate and a voltage of said current supply sufficient to cause bonding between said substrate and said cover chip.

15. A nanochannel system for DNA sequencing comprising:
(a) a substrate, wherein said substrate comprises at least one electrode and a nanochannel having a first end and a second end, wherein said first end of said nanochannel is negatively-charged and said second end of said nanochannel is positively-charged; and
(b) a cover chip, wherein said cover chip is bonded to said substrate.

16. A method of DNA sequencing using a nanochannel system that comprises a substrate comprising at least one pair of electrodes dissected by a nanochannel having a first end and a second end, wherein an inlet reservoir is joined to said first end of said nanochannel and an outlet reservoir is joined to said second end of said nanochannel, the method comprising the steps of:
(a) placing a DNA molecule comprising at least one base in said inlet reservoir;
(b) applying a positive bias voltage to said outlet reservoir and a negative bias voltage to said inlet reservoir sufficient to cause said DNA molecule to be electrically pulled through said nanochannel;
(c) measuring the transverse electrical current between said at least one pair of electrodes as said DNA molecule is pulled through said nanochannel; and
(d) determining the composition of said at least one base in said DNA molecule based on said transverse electrical current.
